(19) United States
(12) Patent Application Publication
(10) Pub. No.: US 2016/0136110 A1
(43) Pub. Date: May 19, 2016

(54) Method for Treating a Neurodegenerative Event

(71) Applicant: MAKScientific, LLC, Mystic, CT (US)

(72) Inventor: Alexandros Makriyannis, Watertown, MA (US)

(21) Appl. No.: 14/941,821

(22) Filed: Nov. 16, 2015

Related U.S. Application Data
(63) Continuation of application No. 13/859,292, filed on Apr. 9, 2013.
(60) Provisional application No. 61/621,756, filed on Apr. 9, 2012.

(51) Int. Cl. A61K 31/12

(52) U.S. Cl. CPC A61K 31/12

(57) ABSTRACT

A method of neuroprotection or treating or healing a wound, orifice, surgical site or a neurodegenerative event in an individual or animal via modulation of at least one neurotrophin by administering a therapeutically effective amount of at least one fatty acid amide hydrolase (FAAH) inhibitor or a physiologically acceptable salt thereof to the individual or animal. Administration of the at least one FAAH inhibitor may be via release from an article of manufacture that includes a support.

### CROSS-REFERENCE TO RELATED APPLICATION

This application is a continuation of U.S. patent application Ser. No. 13/859,292 for Composition and Method for Treating a Neurodegenerative Event, filed Apr. 9, 2013, which claims priority from U.S. Provisional Application No. 61/621,756 filed Apr. 9, 2012, the entire contents of each of which are hereby incorporated by reference.

### STATEMENT OF UNITED STATES GOVERNMENT SUPPORT

The subject matter of the present application was made with United States Government support from the Small Business Innovation Research (SBIR) program under contract number 1R43DA023737. Accordingly, the United States Government may have certain rights to the subject matter of the present application.

### FIELD OF THE INVENTION

The present invention is in the field of neurodegenerative diseases, in particular aimed at preventing, treating and/or reducing symptoms associated with a neurodegenerative event. In one aspect, the invention relates to a method of increasing levels of at least one neurotrophin in vivo to treat a neurodegenerative disease.

### BACKGROUND

It is known that neurotrophins support the survival, development, and function of neurons. Neurotrophins include structurally related factors such as nerve growth factor (NGF), brain-derived neurotrophic factor (BDNF), neurotrophin-3 (NT-3), and neurotrophin-4 (NT-4), and the GDNF (glial cell derived neurotrophic factor) family of ligands (e.g., neurotrurin, artenin and persephin) and ciliary neurotrophic factor (CNTF).

BDNF, for example, acts on certain neurons of the central nervous system (CNS) and the peripheral nervous system (PNS), helping to support survival of existing neurons, and encourage the growth and differentiation of new neurons and synapses (Nature 374 (6521): 450-3 and Annu. Rev. Neurosci. 24: 677-736). There are reports that BDNF is active in certain brain regions such as the hippocampus, cortex, and basal forebrain. Although many brain neurons are formed prenatally, parts of the adult brain are said to retain the ability to grow new neurons from neural stem cells in a process known as neurogenesis. Neurotrophins such as BDNF are understood to help to stimulate and control neurogenesis.

There are reports showing links between changes in the levels and activities of BDNF and a number of neurodegenerative events, such as depression, schizophrenia, obsessive-compulsive disorder, stroke, seizures, toxin exposure, ischemia, hypoxia, traumatic brain injury, multiple sclerosis, spinal cord injury, Alzheimer's disease, Parkinson's disease, Huntington's disease, Rett syndrome and dementia, as well as anorexia nervosa and bulimia nervosa. Increased levels of BDNF in the ventral tegmental area can induce an opiate-dependent-like reward state in naive rats (Science. 2009; 324(5935):1732-4).

Glial cell-derived neurotrophic factor (GDNF), is a protein that potently promotes the survival of many types of neurons. A notable property of GDNF is its ability to support the survival of dopaminergic and motor neurons. These neuronal populations die in the course of Parkinson's disease and amyotrophic lateral sclerosis (ALS). GDNF also regulates kidney development and spermatogenesis.

It is also known that the blood-brain barrier (BBB) poses an obstacle to administering drugs, particularly to areas of the brain. For example, the BBB has been reported to restrict entry of many large and/or hydrophilic molecules from the bloodstream into the brain. Methods of circumventing the BBB include use of carrier-mediated transporters such as glucose and amino acid carriers; receptor-mediated transcytosis for insulin or transferrin; and/or blocking active efflux transporters such as p-glycoprotein. Additional methods include intracerebral implantation (such as with needles) and convection-enhanced distribution. However, each of these methods poses challenges that can severely complicate widespread use by patients and caregivers.

It would be desirable to have a method of modulating the activity of at least one neurotrophin, and more particularly, a method of increasing the activity of at least one endogenous neurotrophin within the CNS of an animal. It would also be desirable to have a method of preventing, treating or reducing the severity of a neurdegenerative event in an animal that includes increasing activity of the neurotrophin within the brain that circumvents permeability challenges posed by the BBB.

### BRIEF DESCRIPTION OF THE DRAWINGS

FIG. 1A shows the in vivo effects of The FAAH inhibitor Compound 1 on GDNF levels; and

FIG. 1B shows the in vivo effects of The FAAH inhibitor Compound 1 on BDNF levels.

FIG. 2 shows the in vitro effects of the FAAH inhibitor, Compound 1, on BDNF levels, alone.

### SUMMARY

Disclosed in the present invention is an article of manufacture that includes a support with a therapeutically effective amount of at least one fatty acid amide hydrolase (FAAH) inhibitor or a physiologically acceptable salt thereof. The article has a wide variety of uses and applications including use to release into an orifice, surgical site, wound (e.g., a burn or other topical injury) the FAAH inhibitor to modulate (increase or decrease, preferably increase) levels of at least one neurotrophin therein.

In a certain embodiment, the article of manufacture is defined as a pharmaceutical preparation and/or a medical product.

In another embodiment, the disclosure provides a method for preventing, treating, and/or reducing symptoms of a neurodegenerative event in a subject. In one embodiment, the method includes administering a therapeutically effective amount of at least one fatty acid amide hydrolase (FAAH) inhibitor or a physiologically acceptable salt thereof, to the subject in need to prevent, treat and/or reduce symptoms of the neurodegenerative event.

In yet another embodiment, the disclosure provides a method of increasing expression of at least one neurotrophin in a cultured cell or tissue. The method includes contacting the cell or tissue with at least FAAH inhibitor or a physiologically acceptable salt thereof sufficient to increase expression of the neurotrophin in the cultured cell or tissue.

### DETAILED DESCRIPTION

As used herein, “neurotrophin” means a mammalian brain derived neurotrophic factor (BDNF) or glial cell derived neurotrophic factor (GDNF), for example, a rodent BDNF, rodent GDNF, human BDNF or human GDNF as well as allelic variants thereof. As used herein, “at least one neurotrophin” or similar phrase means less than five of such neurotrophins, such as one or two.

As used herein, “fatty acid amide hydrolase (FAAH) inhibitor” means a compound that includes a ketone group and has a molecular weight of less than about 1000 D, for example between about 250 to 600 D. FAAH inhibitor compounds for use within the invention further include a urea derivative, a carbamate derivative, a saccharine or a substituted saccharine derivative, a sulphonyl halide derivative, a difluoroketoheterocycle derivative, a trifluoromethylketone derivative or a ketoheterocycle derivative. Particular FAAH inhibitors further include one or more of the following groups to activate the ketone group: dihalo, trihalo, dihaloalkyl. As used herein, “alky” means methyl, ethyl, propyl or isopropyl, trihaloalkyl. As used herein, “halo” means bromo, chloro, or fluoro. As used herein, “alky” means methyl, ethyl, propyl or isopropyl. One particular ketone activating group is a difluoro or trifluoromethyl group. Additional FAAH inhibitors for use within the disclosed product and method optionally include an alpha-keto heterocycle group. As used herein, “heterocycle” means a carbon ring that includes at least one, two or three of a nitrogen, oxygen or sulfur atom the same or different, preferably having five or six ring members. A representative heterocycle is an oxadiazole such as 1,3,4-oxadiazole.

A “urea derivative” refers to compounds having the general formula

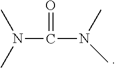

A “carbamate derivative” refers to compounds having the general formula

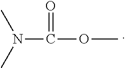

A “sulphonyl halide” refers to compounds having the general formula —SO₂—X wherein X is F, Cl, Br, I.

A “ketoheterocycle” refers to compounds having the general formula

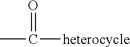

wherein the heterocycle can be attached to the carbonyl group at any position.

A “difluoroketoheterocycle” refers to compounds having the general formula

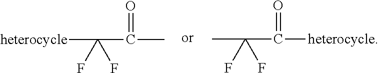

A “trifluoromethylketone” refers to compounds having the general formula —COCF₃.

A “saccharine or a substituted saccharine derivative” refers to compounds having the general formula

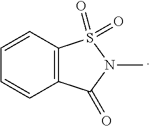

FAAH inhibitor compounds for use within the invention include reversible inhibitors and irreversible inhibitors.

An “reversible inhibitor” refers to compounds such as a trifluoromethylketone derivative, difluoroketoheterocycle derivative, ketoheterocycle derivative or a saccharine derivative.

An “irreversible inhibitor” refers to compounds such as a urea derivative, carbamate derivative or a sulphonyl halide derivative.

As used herein, “at least one FAAH inhibitor” or like phrase means less than five of such inhibitors, such as one, two, three or four of such inhibitors, preferably one or two FAAH inhibitors.

As used herein, “support” as it pertains to an article of manufacture means a composition that provides or is capable of providing at least one FAAH inhibitor to a subject in need thereof. Particular examples of such supports include a stent, depot, matrix, polymer, nanoparticle or combination thereof that is designed to deliver a suitable small molecule to a subject, cell or tissue in need thereof. Examples of suitable supports and methods of making and using the same include, without limitation, those disclosed by Singh R. et al. (2009) Exp. & Mol. Pathology, 86: 215; Hutchinson, F. G. et al. (1990) J. of Controlled Release, 13: 279 as well as U.S. Patent Application Publication Nos. 2010/0023115 (now U.S. Pat. No. 7,951,193); 2010/0023116, 2006/0240059 and U.S. Pat. Nos. 7,135,038 and 7,144,422. In one embodiment, the article of manufacture is a stent that is designed to elute a desired amount of at least one FAAH inhibitor to a patient in need thereof.

As used herein, the conjunction “and” is intended to be inclusive and the conjunction “or” is not intended to be exclusive unless otherwise indicated. For example, the phrase “or, alternatively” is intended to be exclusive. “Or” may be exclusive when describing chemical substitution patterns.

As used herein, “the subject” means any species to which a treatment is or has been treated and which is capable of responding to that treatment. Examples include a mammal such as a domesticated animal (dog, horse, cat, rabbit, sheep, pig, etc.). A particular example of a subject is a human patient that is, will be or is suspected of having a neurodegenerative event and/or is in need of an increased level of at least one neurotrophin.

As used herein, “neurodegenerative event” means stroke, seizure, toxin exposure, ischemia/reperfusion injury, hypoxia, traumatic brain injury, multiple sclerosis, spinal cord injury, Rett syndrome, Alzheimer's disease, Parkinson's disease, Huntington's disease, dementia (HIV related or not), or other neurodegenerative disorders as well as depression, schizophrenia, obsessive-compulsive disorder, anorexia nervosa and bulimia nervosa and others. The term “neurodegenerative event” is also meant to encompass conditions resulting in significant tissue damage that are or may be associated with damage to nerves in the PNS. Illustrative examples include a topical injury such as a bruise, wound or burn, or nerve damage associated with a cut or surgery.

As used herein, “pharmaceutically acceptable salt” means salts with pharmaceutically acceptable acids or bases. Pharmaceutically acceptable salts are well known in the art and described in, for example, S. M. Berge et al. describes pharmaceutically acceptable salts in detail in J. Pharmaceutical Sciences, 66: 1 (1977). Such salts can be prepared in situ during the final isolation and purification of the catechol-based precursor, or separately by reacting the free base function with a suitable organic acid. Examples of pharmaceutically acceptable salts include, without limitation, nontoxic acid addition salts are salts of an amino group formed with inorganic acids such as hydrochloric acid, hydrobromic acid, phosphoric acid, sulfuric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Pharmaceutically acceptable salts of carboxylates and other oxo-acids can be formed with cationic species such as alkali or alkaline earth metal ions including sodium, lithium, potassium, calcium, magnesium, and the like. Further, pharmaceutically acceptable salts include, when appropriate, nontoxic ammonium, quaternary ammonium, and amine cations as well as natural product cations such as choline and acetyl choline and the like. Anionic counterions include halides, hydroxide, carboxylate, sulfate, phosphate, nitrate, alkyl (having from 1 to 6 carbon atoms) sulfonate and aryl sulfonate.

Administering a fatty acid amide hydrolase (FAAH) inhibitor to an animal subjected to experimental nerve damage can be neuroprotective (i.e., decelerate or halt the loss of neurons. See Karanian D A, et al (2005) J Neuroscience 25:7813; and Karanian D A, et al. (2007) J Pharmacol Exp Ther 322:1059. It has been found that at least some of the neuroprotective activity initiated by the FAAH inhibitor is due to an increase in the level of at least one endogenous neurotrophin in the animal. It is believed that the increase in the endogenous neurotrophin afforded by the FAAH inhibitor assists in protecting nerves against damage. As disclosed herein, using at least one FAAH inhibitor provides a new and convenient method of increasing endogenous neurotrophin levels in an animal in need thereof. Significantly, the disclosed product and method allow increasing levels of at least one neutrophin within the CNS of an animal (particularly the brain) without requiring moving a large synthetic neurotrophin or neurotrophin-expressing cells across the BBB. Thus, the disclosed product and method provide a more convenient method of enhancing endogenous neurotrophin levels in the brain by administering smaller FAAH inhibitors to the animal to increase levels of the neurotrophin.

For many applications, it will be useful to pre-select a FAAH inhibitor that can readily cross the BBB into the brain for use with the invention methods. Thus in one embodiment, a FAAH inhibitor with a significant ability to cross the BBB is identified and chosen, thereby providing a corresponding increase in ability to enhance neurotrophin in the brain. However in another embodiment, a FAAH inhibitor is identified and chosen with less BBB permeability, thereby intentionally providing less of an increase in the neurtrophin enhancing capability. Identifying and choosing of molecules with a particular BBB permeability is typically guided by understood parameters such as the condition to be treated and the magnitude of neurotrophin increase that is desired. Known methods are disclosed in Marsicano G, et al. (2003) Science 302:84-88; and Khaspekov L G, et al. (2004) Eur J Neurosci 19:1691-1698.

Practice of the disclosed method can avoid complex and occasionally controversial approaches to enhance neurotrophin levels in animals in need thereof, including direct administration of synthetic neurotrophins or pluirpotent cells such as stem cells. In contrast, disclosed method allows increasing endogenous levels of the neurotrophin and does not require use of synthetic neurotrophins or cells that express the protein. Accordingly, the method provides an indirect but important way of enhancing levels of at least one endogenous neurotrophin that is more convenient and potentially safer than known methods. Known problems and obstacles associated with getting large molecules and cells into the CNS are avoided in many instances.

In one aspect, the disclosure provides an article of manufacture that includes a support comprising a therapeutically effective amount of at least one fatty acid amide hydrolase (FAAH) inhibitor or a physiologically acceptable salt thereof. The article of manufacture has a wide variety of uses and applications including use to release the FAAH inhibitor into an orifice, wound or surgical site the FAAH inhibitor to modulate (particularly increase) at least one endogenous neurotrophin therein. In one embodiment, the article of manufacture is used to stimulate the growth and/or repair of a nerve within the PNS. In another embodiment, the article of manufacture is used to stimulate the growth and/or repair of a nerve within the central nervous system (e.g., brain, spinal cord).

FAAH inhibitors of the disclosure that feature a trifluoromethyl group as the ketone activating group will on occasion be referred to as a “trifluormethyl ketone compound.” Compound 1 is an illustrative example of this class of compounds. Other FAAH inhibitors that include a difluoro group and an alpha-keto heterocycle group consisting of an oxadiazole such as 1,3,4-oxadiazole will sometimes be referred to herein as an “activated diflouromethyl ketone.”

A preferred activated ketone compound for use in the inventive product and method shows inhibition of the rat and/or human FAAH enzyme according to an assay prepared as in Rahn, E et al. (2011) Pharm. Biochem and Behavior 98: 493. Further preferred compounds for use in the disclosed product and method have an IC₅₀ less than about 250 nM, preferably less than 150 nM, and more preferably between from about 0.1 to about 100 nM in the rat or human FAAH inhibition assay. Particular FAAH inhibitor compounds for use with the invention include those disclosed in the following references: Deng, H (2010) Expert Opin. Drug Discovery 5(10): 961-993 and Blankman (2013) Pharmacol Rev 65:849-871, Seierstad (2008) J. Med. Chem., 51 (23), pp 7327-7343, Ahn (2009) Expert Opin. Drug Discov. 4(7):763-784 and references cited therein. Further suitable FAAH inhibitors may also inhibit a rat and/or human monoacylglycerol lipase (MGL) with an IC₅₀ less than about 2500 nM, for instance less than 1000 nM such as between from about 0.1 to about 250 nM in a MGL assay disclosed in Naidoo, V et al. (2011) J. Mol. Neurosci 43:493) using the rat or human enzyme. Such inhibitors will on occasion be referred to herein as “dual FAAH/MGL inhibitors.” According to these assays, Compound 1 being (5-(4-(benzyloxy)phenoxy)-1,1,1-trifluoropentan-2-one) is a FAAH inhibitor (IC₅₀=42 nM).

Additional FAAH inhibitors for use herein (including the activated diflouromethyl ketones described above) are disclosed in International Publication Nos. WO2007/061862, WO2007/14005, WO2008/013963 (now US2009/0306016, disclosing, for instance, suitable difluoroketo analogs and difluoroketo heterocycle analogs for use with the invention), WO2009/052320, and U.S. Patent Application Publication Nos. 2010/0234379, 2010/0261674 (now U.S. Pat. No. 8,293,724), and U.S. Pat. No. 6,462,054, and references disclosed therein. US2009/0306016 is hereby incorporated by reference in its entirety, including the trifluoromethyl ketones and activated difluoromethyl ketones disclosed therein.

The illustrative FAAH inhibitor for use in the inventive step is disclosed in Naidoo, V et al. (2011), supra and WO/2008/013963. In embodiments wherein increasing neurotrophin activity in the CNS is useful, FAAH inhibitors that penetrate the BBB using intravenously administered doses up to 1 mg/kg are identified and selected according to known reliable methods.

Choice of a particular support to use with the disclosed method and article of manufacture will be informed by recognized parameters such as the neurodegenerative event to be treated, age and/or sex of the patient, and particular FAAH inhibitor selected. However in one embodiment it will often be useful to choose a support that can provide between about 0.1 to 50 mg/kg of the FAAH inhibitor.

In one embodiment, the composition includes a support which further includes an adhesive surface adapted to provide contact between the support and the orifice, wound or surgical site in need of the increase of the neurotrophin. The support may also include one or more of a surgical dressing or polymer adapted to retain the FAAH inhibitor or a combination of FAAH inhibitors therein.

In one aspect, the disclosure provides a method for preventing, treating, and/or reducing symptoms of a neurodegenerative event in a subject. In one embodiment, the method includes administering a therapeutically effective amount of at least one fatty acid amide hydrolase (FAAH) inhibitor or a physiologically acceptable salt thereof, to the subject to prevent, treat and/or reduce symptoms of the neurodegenerative event in the subject. In one embodiment, the method further includes the step of increasing the amount of at least one neurotrophin in the central nervous system (CNS) of the subject. A therapeutically effective amount of the FAAH inhibitor will be between 0.1-40.0 mg/kg, for example, preferably between 0.5-30.0 mg/kg. Range parameters may differ depending on the neurodegenerative disease being address, the species to which the FAAH inhibitor is being administered, the gender, the general condition of the subject and other factors recognized in the field.

The particular route of administering the FAAH inhibitor to a subject may vary according to understood parameters in the field such as the condition to be treated, age and gender. However, administration routes such as oral, intradermal, subcutaneous, nasal, intravenous, intramuscular, intrathecal, intraperitoneal, intravaginal or buccal administration will be useful for many applications.

It may not be possible to monitor a neurotrophin in the CNS and particularly the brain without subjecting the subject to discomfort or risk of injury or infection. Although such methods exist (i.e., immunological analysis of CNS fluid using antibodies that detectably bind neurotrophins), it will often be preferred to monitor the increase in neurotrophins indirectly, for example, by detecting the growth of new nerves and nervous tissue. In one embodiment, the method described herein further includes the step of detecting the growth of new nerves in the subject as indicative of the increase in the levels of the neurotrophin afforded by the FAAH inhibitor. Such a step can be achieved by one or a combination of recognized methods including subjecting the subject to a neuroimaging technique and analyzing the results. In one embodiment, the neuroimaging technique may be selected from one or more of computed tomography (CT) and magnetic resonance imagining (MRI).

In another aspect, the disclosure provides a method of increasing expression of at least one neurotrophin in a cultured cell or tissue. In one embodiment, the method includes contacting the cell or tissue with at least one fatty acid amide hydrolase (FAAH) inhibitor or a physiologically acceptable salt thereof sufficient to increase expression of the neurotrophin in the cultured cell or tissue. Such a method may also include the step of detecting the increase in neurotrophin expression such as by use of a recognized immunological assay (i.e., Western blot, RIA, etc.).

N-arachidonylethanolamine (AEA) is a member of a class of bioactive lipids called fatty acid amides. AEA has been implicated in neuroprotective on-demand responses and found to increase in response to neuroinjury. Fatty acid amide hydrolase (FAAH) is an integral membrane hydrolase with a single N-terminal transmembrane domain. FAAH is the principal catabolic enzyme for hydrolyzing the bioactive lipids and deactivating them. Thus inhibiting the enzyme that hydrolyses the bioactive fatty acid amides provides for the maintenance of elevated levels of the amide allowing for protection of injured neutrons.

As described herein, the neurotrophins, BDNF and GDNF, act on certain neurons of the central nervous system and the peripheral nervous system, helping to support the survival of existing neurons, and encourage the growth and differentiation of new neurons and synapses.

Kainic acid (KA) administration induced seizures and the same neurodegenerative events exhibited in vitro. KA caused calpain-mediated spectrin breakdown, declines in synaptic markers, and disruption of neuronal integrity in cultured hippocampal slices.

The FAAH inhibitor may be delivered to the subject in a pharmaceutical composition containing other pharmaceutically acceptable components, such as for example, vehicles, buffer, surfactants, emulsifiers, anticoagulants, and the like. Vehicles include, for example, pharmaceutically acceptable solvents such as water, saline, ethanol, glycerine and the like.

Accordingly, provided herein is a method to pharmacologically modulate (particularly increase) the FAAH to provide sustained enhancement of at least one neurotrophin. In one embodiment, a method for treating neurodegenerative events in a subject includes administering a therapeutically effective amount of a material selected from one of a FAAH inhibitor that promotes neurotrophin expression, or a physiologically acceptable salt thereof, to the subject, in which the neurodegenerative events include those associated with stroke, seizures, toxin exposure, ischemia, hypoxia, traumatic brain injury, multiple sclerosis, spinal cord injury, Alzheimer's disease, Parkinson's disease, Huntington's disease, and other neurodegenerative disorders.

As will become evident, various modifications and enhancements of the above embodiments are within the scope of the subject matter disclosed and claimed herein. The following Examples are intended to illustrate but not limit the scope of the present invention.

### Example 1

Young rats (PND 22) were injected i.p. with kainic acid (KA), followed immediately by vehicle (veh) or the FAAH inhibitor, Compound 1 (8 mg/kg, i.p.). After 24 h, hippocampus was rapidly dissected, and the homogenates were centrifuged to obtain supernatant for ELISA measures of GDNF) and BDNF. Standard curves were used to convert measures to pg cytokine per mg supernatant protein (±SEM). Unpaired t-tests: GDNF, *p<0.02; BDNF, ***p<0.001.

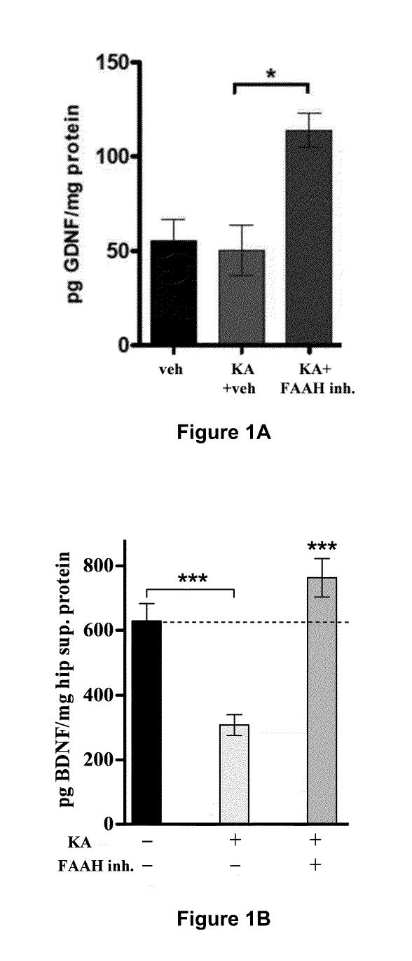

The FAAH inhibitor, Compound 1, enhances GDNF levels (FIG. 1A) and also protects against excitotoxin-induced reduction in BDNF (FIG. 1B). The data shows that enhancing cytokine components of cellular repair responses will promote protection against convulsant-mediated brain damage.

### Example 2

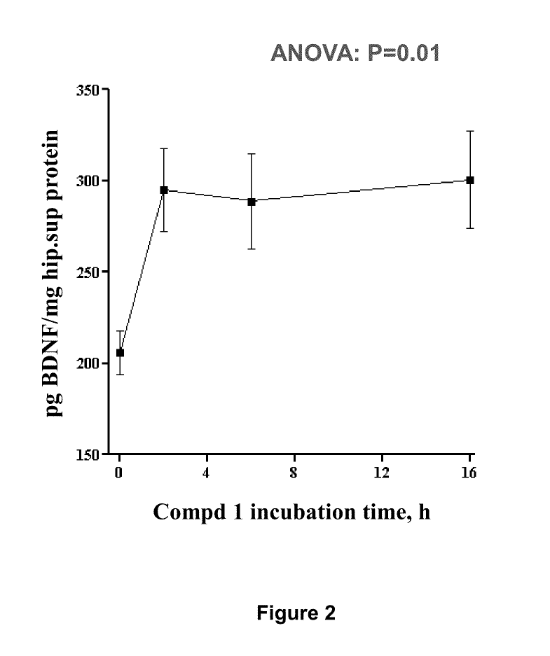

In an in vitro experiment using hippocampal slice cultures and the FAAH inhibitor, Compound 1 alone, the inhibitor Compound 1 was applied at 30 μM to hippocampal slice cultures and groups of slices were harvested over time to assess tissue homogenates by the BDNF ELISA test kit. The results are shown in FIG. 2.

Although the present invention has been shown and described with reference to particular examples, various changes and modifications which are obvious to persons skilled in the art to which the invention pertains are deemed to lie within the spirit, scope and contemplation of the subject matter set forth in the appended claims. The disclosure of all documents referred to herein is incorporated by reference.

What is claimed is:

1. A method of treating or preventing a neurodegenerative event comprising modulating at least one neurotrophin in an individual or animal by administering at least one fatty acid amide hydrolase (FAAH) inhibitor or physiologically acceptable salt thereof to said individual or animal.

2. The method of claim 1, comprising modulating at least one neurotrophin located in the central nervous system (CNS) or the peripheral nervous system (PNS) of the individual or animal.

3. The method of claim 1, comprising neuroprotection or treating or preventing a neurodegenerative event associated with one or more of the group consisting of stroke, seizure, toxin exposure, ischemia/reperfusion injury, hypoxia, traumatic brain injury, multiple sclerosis, spinal cord injury, Rett syndrome, Alzheimer's disease, Parkinson's disease, Huntington's disease, other neurodegenerative disorders, as well as depression, schizophrenia, obsessive-compulsive disorder, anorexia nervosa and bulimia nervosa, a wound, burn, bruise, cut or surgery.

4. The method of claim 1, comprising repairing damaged nerves or detecting growth of new nerves.

5. The method of claim 1, comprising increasing Glial cell-derived neurotrophic factor or Brain-derived neurotrophic factor.

6. The method of claim 1, wherein at least one FAAH inhibitor is administered to said individual or animal via release from an article of manufacture comprising a support.

7. A method of increasing expression of at least one neurotrophin in a damaged tissue or cultured cell or tissue, comprising contacting the cell or tissue with at least one fatty acid amide hydrolase (FAAH) inhibitor or a physiologically acceptable salt thereof.

8. A method of increasing levels of at least one neurotrophin in an individual or animal by administering at least one fatty acid amide hydrolase (FAAH) inhibitor or physiologically acceptable salt thereof to said individual or animal.

9. The method of claim 9, comprising increasing Glial cell-derived neurotrophic factor or Brain-derived neurotrophic factor.
